(19) United States
(12) Patent Application Publication
(10) Pub. No.: US 2005/0123974 A1
(43) Pub. Date: Jun. 9, 2005

(54) Methods and compositions relating to single reactive center reagents

(75) Inventors: Rudolf Gilmanshin, Waltham, MA (US); Amie Jo Hatch, Worcester, MA (US)

Correspondence Address: WOLF GREENFIELD & SACKS, PC;FEDERAL RESERVE PLAZA, 600 ATLANTIC AVENUE, BOSTON, MA, 02210-2211, US

(73) Assignee: U.S. Genomics, Inc., Woburn, MA (US)

(21) Appl. No.: 10/991,964

(22) Filed: Nov. 17, 2004

Related U.S. Application Data
(60) Provisional application No. 60/520,927, filed on Nov. 17, 2003.

(51) Int. Cl. H01L 21/00; C12Q 1/68; G01N 33/53

(52) U.S. Cl. 435/6; 435/7.5; 438/1

(57) ABSTRACT

Methods of preparing single reactive center reagents are encompassed by the invention. The invention also includes compositions of single reactive center reagents and methods of use thereof for labeling and analyzing polymers such as nucleic acids.

### RELATED APPLICATIONS

This application claims priority to U.S. Provisional Application Ser. No. 60/520,927, entitled “SINGLE CENTER QUANTUM DOTS FOR FLUORESCENT TAGGING”, filed Nov. 17, 2003, the entire contents of which are incorporated by reference herein.

### FIELD OF THE INVENTION

The invention provides single reactive center reagents, methods for generating single reactive center reagents from multi reactive center reagents, and methods of use thereof for analysis of biological molecules including cells and polymers.

### BACKGROUND OF THE INVENTION

Various research reagents are known which have multiple reactivities. This is due to the manufacture of such reagents which generally is geared towards creating as many reactive sites on a given reagent as possible. Such reagents include compounds used to bind and/or label biological molecules, and examples include particles and beads that are derivatized on their surface, usually by their manufacturer for ease of use in the field. One particular example is quantum dots which are commercially available with, for example, streptavidin conjugated to their surface. While such reagents are useful for a number of applications, their use in other applications, particularly those requiring single reactivities, for example, is limited if not altogether impeded. There exists a need to transform such multiple reactive center reagents into single reactive center reagents to be used in a number of biological applications.

### SUMMARY OF THE INVENTION

The invention provides in a broad sense methods for producing single reactive center reagents, the reagents themselves, and methods of using these reagents for analyzing molecules.

In one aspect, the invention provides a method for producing a single reactive center reagent comprising contacting a multi reactive center reagent having a plurality of first reactive groups with a) a probe conjugated to a second reactive group that is reactive to the first reactive group, and b) unconjugated second reactive group, under conditions that favor binding of none or one conjugated probe (i.e., a probe conjugated to a second reactive group) per reagent.

Various embodiments relate to the various aspects recited herein. Some of these embodiments are recited below and it is to be understood that they apply equally to the various aspects of the invention.

In one embodiment, the multi reactive center reagent is inherently detectable. The multi reactive center reagent may be a quantum dot or a fluorescent bead, for example. In another embodiment, the multi reactive center reagent is not inherently detectable. The multi reactive center reagent may be a protein, a bead, or a particle, for example.

In one embodiment, the multi reactive center reagent inherently comprises the plurality of first reactive groups. An example is a protein or peptide having amino acids with side chains having reactive groups (e.g., amines, carboxylic acids, etc.). In another embodiment, the multi reactive center reagent is derivatized to comprise the plurality of first reactive groups. Examples include quantum dots coated with streptavidin or biotin. The first reactive groups and second reactive groups may be selected from the group consisting of biotin, streptavidin reactive groups, aptamers, aptamer ligands, receptors, receptor ligands, nucleic acids, enzymes, substrates, amines, carboxylic acids, esters, amides, carbonyls, alcohols and cyanos, but they are not so limited. In one embodiment, the first reactive group is biotin and the second reactive group is a streptavidin reactive group (i.e., a biotin binding site) or an avidin reactive group (i.e., a biotin binding site). In another embodiment, the first reactive group is a streptavidin reactive group or an avidin reactive group and the second reactive group is biotin. In yet another embodiment, the first reactive group is an antigen or hapten and the second reactive group is an antibody reactive group (i.e., a single antigen binding site from an antibody). The antibody reactive group may also be an antibody fragment having a single antigen binding site (e.g., an Fab fragment). Alternatively, the first reactive group may be an antibody (or antibody fragment) reactive group and the second reactive group may be an antigen or hapten. In still other embodiments, the first reactive group is a receptor and the second reactive group is a receptor ligand; or the first reactive group is a receptor ligand and the second reactive group is a receptor; or the first reactive group is an aptamer and the second reactive group is an aptamer ligand; or the first reactive group is an aptamer ligand and the second reactive group is an aptamer; or the first reactive group is an amine and the second reactive group is an ester; or the first reactive group is an ester and the second reactive group is an amine.

The first reactive groups and second reactive groups may interact reversibly. For example, the first reactive groups and second reactive groups may interact by hydrogen bonding, ionic bonding and Wan der Waals forces. Alternatively, the first reactive groups and second reactive groups may interact irreversibly. For example, the first reactive groups and second reactive groups may interact covalently.

In one embodiment, the probe is an antibody or antigen-binding fragment thereof, an antigen, an aptamer, an aptamer ligand, a nucleic acid, an enzyme, a substrate, a receptor or a receptor ligand.

In one embodiment, the probe is a nucleic acid probe. The nucleic acid probe may be comprised of DNA, RNA, PNA, LNA, or combinations thereof. It may have a length of at least 5 nucleotides, at least 10 nucleotides, at least 15 nucleotides, at least 20 nucleotides, or at least 25 nucleotides. In some embodiments, the nucleic acid probe comprises a linker when conjugated.

In one embodiment, the conditions that favor binding of none or one conjugated probe per reagent comprise excess unconjugated second reactive group. Excess unconjugated second reactive group may represent a concentration that is at least 10-fold, at least 100-fold, at least 1000-fold, at least 10⁴-fold, or at least 10⁵-fold greater than the concentration of second reactive groups conjugated to the probe.

In other embodiments, the conditions that favor binding of none or one conjugated probe per reagent comprise reducing binding time, increased temperature, or altered ion (e.g., salt) concentration.

In one embodiment, the multi reactive center reagent having a plurality of first reactive groups is first contacted with the probe conjugated to a second reactive group under conditions that favor binding of none or one probe conjugated to a second reactive group per reagent, and then contacted with excess unconjugated second reactive group.

In another embodiment, the multi reactive center reagent having a plurality of first reactive groups is contacted with the probe conjugated to a second reactive group and excess unconjugated second reactive group simultaneously.

In still another embodiment, the multi reactive center reagent having a plurality of first reactive groups is first contacted with excess unconjugated second reactive group, and then contacted with the probe conjugated to a second reactive group.

The method may further comprise separating reagents bound by one second reactive group from those bound by none or more than one second reactive group. Such separating may be accomplished by size separation, using approaches such as electrophoresis or size exclusion chromatography. Such separation may also be accomplished by charge separation, using approaches such as electrophoresis or ion-exchange chromatography. Such separating may also be accomplished magnetically.

In embodiments in which the reagent is a quantum dot, the quantum dot may be a CdSe quantum dot, a PbSe quantum dot, an InP quantum dot, an InAs quantum dot, or a CdTe quantum dot. The quantum dot may emit in the ultraviolet range, the visible range, the red to near infrared range, or the near infrared range. In one embodiment, the quantum dot emits at about 480 nm, about 520 nm, about 630 nm or about 660 nm. In one embodiment, the quantum dot is excited electronically. In another embodiment, the quantum dot is excited by a laser, arc, lamp source or LED.

In one embodiment, the unconjugated second reactive groups are unconjugated to probe but are conjugated to a detectable label. In a related embodiment, the detectable label is an organic fluorophore, which may be a fluorescence resonance energy transfer (FRET) donor or a FRET acceptor, but it is not so limited.

In another aspect, the invention provides a method for producing a single reactive center quantum dot comprising contacting a streptavidin-conjugated quantum dot with a biotin-conjugated nucleic acid probe and unconjugated biotin, under conditions that favor binding of none or one biotin-conjugated nucleic acid probe per quantum dot. In one embodiment, the streptavidin-conjugated quantum dot is first contacted with the biotin-conjugated nucleic acid probe under conditions that favor binding of none or one biotin-conjugated nucleic acid probe per quantum dot, and then contacted with excess unconjugated biotin. In another embodiment, the streptavidin-conjugated quantum dot is contacted with the biotin-conjugated nucleic acid probe and excess unconjugated biotin simultaneously. In yet another embodiment, the streptavidin-conjugated quantum dot is first contacted with the excess unconjugated biotin, and then contacted with the biotin-conjugated nucleic acid probe.

In still another aspect, the invention provides a method for producing a single reactive center quantum dot comprising contacting a biotin-conjugated quantum dot with a streptavidin-conjugated nucleic acid probe and unconjugated streptavidin, under conditions that favor binding of none or one streptavidin-conjugated nucleic acid probe per quantum dot. In one embodiment, the biotin-conjugated quantum dot is first contacted with the streptavidin-conjugated nucleic acid probe under conditions that favor binding of none or one streptavidin-conjugated nucleic acid probe per quantum dot, and then contacted with excess unconjugated streptavidin. In another embodiment, the biotin-conjugated quantum dot is contacted with the streptavidin-conjugated nucleic acid probe and excess unconjugated streptavidin simultaneously. In still another embodiment, the biotin-conjugated quantum dot is first contacted with excess unconjugated streptavidin, and then contacted with the streptavidin-conjugated nucleic acid probe.

In one embodiment, the conditions include a reduced binding time, an increased temperature, or an altered ion (e.g., salt) concentration.

In one embodiment, excess unconjugated biotin is a concentration of unconjugated biotin that is at least 10-fold, at least 100-fold, at least 1000-fold, at least 10⁴-fold, or at least 10⁵-fold greater than the concentration of biotin conjugated to the probe. In another embodiment, excess unconjugated streptavidin is a concentration of unconjugated streptavidin that is at least 10-fold, at least 100-fold, at least 1000-fold, at least 10⁴-fold, or at least 10⁵-fold greater than the concentration of streptavidin conjugated to the probe.

In one embodiment, after contact with the excess unconjugated biotin, the streptavidin-conjugated quantum dots are exposed to conditions that favor limited dissociation of unconjugated biotin from the streptavidin-conjugated quantum dots. In another embodiment, after contact with the excess unconjugated streptavidin, the biotin-conjugated quantum dots are exposed to conditions that favor limited dissociation of unconjugated streptavidin from the biotin-conjugated quantum dots.

The methods may further comprise separating streptavidin-conjugated quantum dots that are bound by one biotin-conjugated oligonucleotide from those bound by none or more than one biotin-conjugated oligonucleotide, or separating biotin-conjugated quantum dots that are bound by one streptavidin-conjugated oligonucleotide from those bound by none or more than one streptavidin-conjugated oligonucleotide. Such separating is described above and herein.

The contemplated attributes of quantum dots and probes are as described above and herein.

In another aspect, the invention provides a composition comprising a single reactive center reagent as produced according to any of the foregoing methods.

In still another aspect, the invention provides a method for analyzing a target molecule comprising contacting a target molecule with the single reactive center reagent as produced by any of the foregoing methods, or the single reactive center quantum dot as produced by any of the foregoing methods and determining a binding pattern of the single reactive center reagent or the single reactive center quantum dot to the target molecule.

In one embodiment, the single reactive center quantum dot is a plurality of single reactive center quantum dots and each of the plurality has a unique emission spectrum. In another embodiment, the single reactive center reagent is a plurality of single reactive center reagents and each of the plurality has a unique emission spectrum.

In one embodiment, the single reactive center quantum dot is not first separated from other quantum dots. In another embodiment, the single reactive center reagent is not first separated from other reagents. In related embodiments, the binding pattern is based on coincident binding events of at least two single reactive center reagents or at least two single reactive center quantum dots.

In still another embodiment, the binding pattern is based on coincident binding events of a single reactive center reagent or a single reactive center quantum dot and a second probe conjugated to an organic fluorophore. The single reactive center reagent or the single reactive center quantum dot may be a donor FRET fluorophore and the organic fluorophore may be an acceptor FRET fluorophore.

In one embodiment, the target molecule is a biological molecule, such as but not limited to a naturally occurring polymer. The target molecule may be a nucleic acid, in some embodiments.

These and other embodiments of the invention will be described in greater detail herein.

Each of the limitations of the invention can encompass various embodiments of the invention. It is therefore anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

The phraseology and terminology used herein is for the purpose of description and should not be regarded as limiting. The use of “including”, “comprising”, or “having”, “containing”, “involving”, and variations thereof herein, is meant to encompass the items listed thereafter and equivalents thereof as well as additional items.

### BRIEF DESCRIPTION OF THE FIGURES

FIG. 1A is a schematic illustrating the excitation and emission spectra of organic fluorophores.

FIG. 1B is a schematic illustrating the excitation and emission spectra of quantum dots.

FIG. 2 is a schematic illustrating quantum dots conjugated to streptavidin reactive groups and bound to a biotin-conjugated oligonucleotide which in turn binds to a target RNA molecule. It is to be understood that this schematic applies equally to other non-quantum dot reagents as well as to other reversible and irreversible reactive groups.

FIG. 3 is a schematic illustrating quantum dots conjugated to streptavidin reactive groups which are first contacted with biotin-conjugated oligonucleotides, and then with excess free (unconjugated) biotin. It is to be understood that this schematic applies equally to other non-quantum dot reagents as well as to other reversible and irreversible reactive groups. The end result is a quantum dot having only one oligonucleotide bound to its surface with all other streptavidin reactive groups bound to free biotin.

FIG. 4 is a schematic illustrating quantum dots conjugated to streptavidin reactive groups which are simultaneously contacted with biotin-conjugated oligonucleotides and excess free biotin. It is to be understood that this schematic applies equally to other non-quantum dot reagents as well as to other reversible and irreversible reactive groups. The vast molar excess of free biotin favors quantum dots having only one surface bound oligonucleotide with all other streptavidin reactive groups bound to free biotin.

FIG. 5 is a schematic illustrating quantum dots conjugated to streptavidin reactive groups first contacted with excess free biotin and then with biotin-conjugated oligonucleotides. Generation of quantum dots having only one surface bound oligonucleotide with all other streptavidin reactive groups bound to free biotin depends upon dissociation of free biotin from the quantum dots, thereby making a reactive group available to the biotin-conjugated oligonucleotide. It is to be understood that this schematic applies equally to other non-quantum dot reagents as well as to other preferably reversible reactive groups.

It is to be understood that the Figures are not required for enablement of the invention.

### BRIEF DESCRIPTION OF THE SEQUENCE LISTING

SEQ ID NO: 1 is the nucleotide sequence of a nucleic acid probe attached to a single reactive center quantum dot.

SEQ ID NO:2 is the nucleotide sequence of a complementary oligonucleotide attached to a magnetic bead.

### DESCRIPTION OF THE INVENTION

In its broadest sense, the invention relates to the creation of single reactive center reagents either de novo or from multi reactive center reagents. The invention also relates to methods of using the single reactive center reagents in a number of applications including but not limited to analyzing biological molecules such as nucleic acids.

As used herein, a reactive center is a reactive group to which a molecule can be conjugated. Such conjugation can be reversible (e.g., a non-covalent interaction between two reactive groups, such as a hydrogen bond, an ionic bond or Wan der Waals forces) or irreversible (e.g., a covalent interaction between two reactive groups). A single reactive center reagent is a compound (i.e., a reagent) having only one reactive center (i.e., it possesses only one reactive group to which a molecule such as a target molecule can be conjugated either reversibly or irreversibly). A multi (or multiple) reactive center reagent is a compound (i.e., a reagent) having more than one (and often times, tens or hundreds) of reactive centers (i.e., it possesses tens or hundreds or more reactive groups to which molecules such as target molecules can be conjugated either reversibly or irreversibly). Multi reactive center reagents may be conjugated to only one type of molecule or may be conjugated to a plurality of molecules. Because of the multiple reactive centers on each, such reagents may be more prone to agglomeration, thereby limiting their utility in some applications.

A reactive center “reagent” is any compound having at least one reactive group. Such reactive groups may be inherent to the compound. Alternatively, the compound may be derivatized to include such reactive groups. An example of a reactive center reagent having inherent reactive groups is a peptide, polypeptide or protein. Various amino acid side chains have reactive groups such as amine groups (e.g., lysine, arginine and histidine) or carboxylic acid groups (e.g., glutamic acid and aspartic acid). Examples of reactive center reagents which are derivatived to include reactive groups include derivatized particles (e.g., magnetic particles), derivatized beads (e.g., magnetic beads, fluorescent beads and polystyrene beads), derivatized quantum dots, and the like. These reactive center reagents can be derivatized to include reactive groups that covalently or non-covalently conjugate to other reactive groups. Examples of reactive groups that can covalently conjugate to other reactive groups (leading to an irreversible conjugation) include but are not limited to amine groups (which react to, for example, esters to produce amides), carboxylic acids, amides, carbonyls (such as aldehydes, ketones, acyl chlorides, carboxylic acids, esters and amides) and alcohols. Those of ordinary skill in the art will be familiar with other “covalent” reactive groups. Examples of reactive groups that non-covalently conjugate to other molecules (leading to a reversible conjugation) include biotin and streptavidin reactive groups (which react with each other), antibody (or antibody fragment) reactive groups and antigens, receptors and receptor ligands, aptamers and aptamer ligands, nucleic acids and their complements, and the like. Virtually any reactive group is amenable to the methods of the invention, provided it participates in an interaction of sufficient affinity to prevent substantial dissociation at later times.

As used herein, a streptavidin reactive group is a site on streptavidin that binds to biotin. There are four biotin binding sites on each streptavidin molecule. Similarly, a biotin reactive group is a site on biotin that binds to streptavidin. An antibody reactive group is a site on an antibody that binds to an antigen. There are two antigen binding sites on each antibody. Antibody fragments useful in the invention are fragments that include an antigen binding site. An example of a such a fragment is the Fab fragment. Single chain antibodies (scFv) which comprise a heavy chain variable region and a light chain variable region that contribute to form one reactive group (or one antigen binding site) can also be used in the invention.

For the sake of convenience, reagents will sometimes be referred to herein as particles, proteins, quantum dots, beads, and the like; however, it is to be understood that such statements apply equally to other forms of reagents as described herein and are not to be interpreted as limiting an aspect or embodiment of the invention.

In one aspect, the invention provides a method for generating a single reactive center reagent. This can be accomplished in a number of ways. Thus, for example, a single reactive center quantum dot can be generated from a multi reactive center quantum dot, such as for example a streptavidin conjugated quantum dot. Such quantum dots are commercially available from for example Quantum Dot Corporation and Evident Technologies, Inc. These dots are estimated to contain tens (e.g., anywhere from 1 to more than a hundred) streptavidin molecules attached to their surface. In one aspect, the invention provides methods for saturating all but one biotin binding sites with excess free biotin, and leaving one streptavidin reactive group available to bind to a probe. As used herein “free biotin” refers to biotin that is not conjugated to a probe and is therefore also interchangeably referred to as unconjugated biotin. However, it is to be understood that such biotin may be conjugated to a detectable label, as described herein. The probe in this example will itself be conjugated to biotin, and is therefore referred to as a biotin-conjugated probe or a biotinylated probe. In an accompanying or alternative embodiment, discussed in greater detail herein, the invention also provides methods for isolating single reactive center quantum dots from dots containing none or more than one reactive center.

It is to be understood that the reactive groups of a multi-reactive center reagent can be the same but are usually different from the reactive group of the single reactive center reagent.

The probe is a molecule that binds to a target of interest. The nature of the probe will depend upon the application and may also depend upon the nature of the target. Preferably, the probe demonstrates greater affinity for its target than for other molecules (e.g., based on the sequence or structure of the target). Probes can be virtually any compound that binds to a target with sufficient specificity. Examples include nucleic acids that bind to complementary nucleic acid targets via Watson-Crick and/or Hoogsteen binding, aptamers which are nucleic acids that bind to nucleic acid targets or non-nucleic acid targets due to structure rather than sequence of the target, aptamer ligands, antibodies, enzymes, enzyme substrates, receptors, receptor ligands, etc. It is to be understood that although many of the exemplifications provided herein are related to nucleic acid probes and nucleic acid targets, the invention is not so limited and other probe and target combinations are envisioned. As an example, a single center reactive quantum dot indirectly conjugated to a newly synthesized aptamer may be used to screen a library or molecules for an aptamer ligand, and vice versa. Other similar applications will be readily envisioned by those of ordinary skill in the art.

Probes are referred to as being “indirectly conjugated” to the single reactive center reagent. This is because such conjugation involves the intermediate interaction of the two reactive groups (i.e., one present on the reagent and one conjugated to the probe).

If the probe is nucleic acid in nature, it may contain naturally occurring elements such as DNA and RNA or non-naturally occurring elements such as PNA and LNA, or combinations thereof, as discussed in greater detail herein.

Various target molecules can be bound by the probes. Virtually any molecule of interest can be a target provided it has a corresponding probe. Thus, target molecules include but are not limited to amino acid based molecules such as peptides, polypeptides and proteins; sugar based molecules such as carbohydrates, saccharides, oligosaccharides and polysaccharides; and nucleic acids such as DNA (e.g., genomic DNA including nuclear DNA and mitochondrial DNA, and cDNA) and RNA (e.g., mRNA, miRNA and siRNA). As used herein, the terms “target” and “target molecule” are used interchangeably.

In one aspect the invention contemplates contacting a multi reactive center reagent such as a quantum dot derivatized with streptavidin with a biotin-conjugated probe and free biotin. The incubation is varied in order to favor the generation of quantum dots with none or few (preferably one) reactive center bound to the biotin-conjugated probe. Factors that favor such an outcome include the relative amounts of each molecule, order of addition of the molecules, incubation time, temperature, salt or other ion concentration, pH, and the like. Preferably, the free biotin is provided in excess. Excess free biotin means a concentration of biotin that exceeds the number of biotin binding sites on the streptavidin molecules by at least 5 and more preferably 10 and that almost outcompetes conjugated biotin for binding to streptavidin. Thus, excess free biotin can be represented as a concentration ratio or fold excess over the concentration of conjugated biotin. In these terms, excess free biotin may be 10-fold more, 100-fold more, 1000-fold more, 10⁴-fold more, 10⁵-fold more, or even more free biotin than conjugated biotin. A similar meaning is imparted to other second reactive groups.

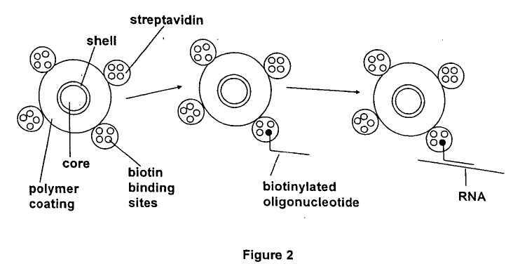

Exemplary methods for making single reactive center quantum dots are shown in FIGS. 2-5. These methods use streptavidin-conjugated quantum dots as the starting multiple reactive center reagent, as shown in FIG. 2. It is to be understood that although the Figures illustrate methods using streptavidin derivatized quantum dots, such methods can just as easily be carried out using biotin-derivatized quantum dots and streptavidin-conjugated probes. It is also to be understood that any multiple reactive center reagent is equally suitable provided that a corresponding reactive group is used in place of biotin.

The quantum dots as purchased from Quantum Dot Corporation have a polymer coating layer that consists of solubilizing detergent armored with a cross-linked polymer on its outer surface (FIG. 2). This outer surface also includes carboxylic acid (—COOH) reactive groups, which are used to conjugate streptavidin to the surface. Addition of the detergent-polymer layer and streptavidin increases the total diameter of the dot by up to about 10-15 nm. Every quantum dot includes several tens of streptavidin molecules and every streptavidin molecule includes 4 biotin binding sites.

To produce a single center quantum dot, the reagent is exposed to a biotin-conjugated probe (such as for example an oligonucleotide). Probes may be directly or indirectly conjugated to second reactive groups such as biotin. Indirect conjugation involves linkers or spacers that link the probe to the second reactive group. In some embodiments, flexible linkers are preferred. Examples of suitable linkers are provided herein. The hybridization reaction is performed so that only one probe is bound per quantum dot (FIG. 2).

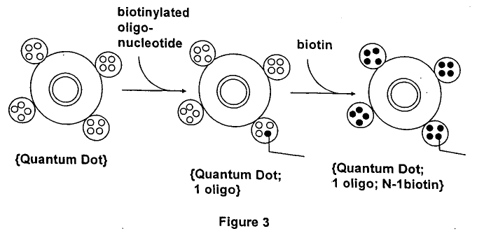

Another way of favoring single reactive center quantum dots is by contacting the dots with a biotin-conjugated probe first, followed by contact with the excess free biotin. The reaction of streptavidin-conjugated quantum dots with a biotinylated probe (such as an oligonucleotide) is quenched at a very early stage (FIG. 3). This results in a mixed population of quantum dots, some having no probe attached and some having only one probe attached. The reaction should be done sufficiently slowly so as to control the timing of the reaction (and thus the amount of probe which has bound to the quantum dot). This can be achieved by decreasing the temperature and/or the concentration of quantum dots or biotinylated probe. The reaction is quenched by addition of an overwhelming amount of free biotin, which effectively outcompetes any remaining biotinylated probe for binding to the quantum dots.

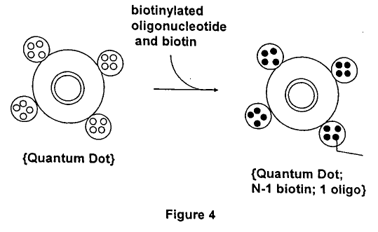

In another embodiment, the dots, excess free biotin and biotin-conjugated probe are contacted and incubated simultaneously (FIG. 4). The ratio of free biotin and the biotinylated probe is adjusted so that quantum dots including only one or no probe are formed as a result of the reaction. The quantum dots that contain no oligonucleotide (or more than one oligonucleotide, as is possible with other embodiments) can be removed afterwards, for example, according to size and/or charge or magnetically, as described in greater detail herein.

In a general sense, each of the afore-mentioned embodiments can be carried out using reversible or irreversible reactive groups such as streptavidin and biotin or amines and esters.

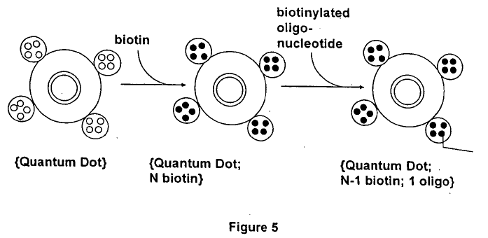

In yet another approach, all biotin-binding sites on streptavidin are saturated with free biotin (FIG. 5), after which the quantum dots are incubated with biotinylated probe. Under some conditions (e.g., elevated temperature, long incubation time, reduced salt concentration, excess cold competitor, etc.), a slow exchange is possible (i.e., a naturally occurring limited dissociation of “free” biotin for the quantum dots and association of biotinylated probe). As used herein, limited dissociation refers to the dissociation of single biotins from the quantum dots. This results is a proportion of quantum dots having a single probe bound thereto. Again, because the reaction is very slow and inefficient, the vast majority of quantum dots includes either one or no probes. This embodiment preferably involves the use of reversible reactive groups such as streptavidin reactive groups and biotin. The quantum dots that contain one probe can be isolated from the population of quantum dots using methods described herein.

It is to be understood that non-probe biotin conjugates can also be used in these any of the foregoing embodiments, provided that such conjugates do not interfere with the hybridization of the probe with its ultimate target. In some embodiments, detectably labeled biotin (e.g., fluorescently labeled biotin) can be used to saturate streptavidin reactive groups or to quench a reaction, provided it does not interfere with probe-target binding.

The invention therefore contemplates conditions that result in none, preferably one, or few (e.g., two or three) reactive center sites being bound by a conjugated probe. The invention further contemplates various methods for isolating single reactive center reagents from reagents having none or more than one reactive center. The exact nature of the isolation method will ultimately depend upon the properties of the reagent and/or the type of reactive groups derivatized thereto. However, generally these methods may include but are not limited to size separation, charge separation and magnetic separation. Size exclusion chromatography or electrophoresis can be used to separate the desired reagents from the other reaction byproducts based at least partly on size. For example, quantum dots increase in size with each additional layer on their surface. Therefore, quantum dots with a single reactive center bound to a probe will differ in size from those having none or more than one reactive center. Ion-exchange chromatography or electrophoresis can be used to separate the desired reagents for the other reaction byproducts based at least partly on charge.

The isolation of reagents bound to a single probe can also be accomplished using magnetic separation, which is dependent on the nature of the probe but essentially independent of size and charge. The magnetic separation is dependent on the probe because it employs a binding partner with affinity for the probe. For example, if the probe is an oligonucleotide, the binding partner could be another oligonucleotide (of identical or different size) having a complementary sequence. The binding partners are themselves provided in the context of a magnetic solid support such as a particle, bead, and the like. As an example, magnetic beads bound to an oligonucleotide that is complementary to the reagent-bound oligonucleotide are used. The reaction mixtures from the various afore-mentioned embodiments are incubated with such beads and hybridization is allowed to occur. Reagents conjugated to such oligonucleotide probes bind to the beads. They can be isolated from the rest of the reaction mixture (including the rest of the reagents) by, for instance, the application of a magnetic field. The quantum dots can be then released from the beads, for example, by heating, decreasing salt concentration, increasing the concentration of “cold” competitor oligonucleotide (i.e., oligonucleotide that competes with the reagent bound oligonucleotide for binding to the magnetic bead, etc.). It is to be understood that this approach can be employed for other probe types such as antibodies, aptamers, etc. provided that a binding partner for each is available and can be conjugated to a magnetic solid support.

The single reactive center reagents of the invention can be used to analyze molecules, including biological molecules such as nucleic acids. As an example, the resulting single reactive center reagent comprising an oligonucleotide probe, as shown for example in FIG. 2, can be used to analyze nucleic acid targets having a sequence complementary to that of the oligonucleotide. An example of this method is presented in more detail in the Examples.

Generally, single reactive center reagents are added in excess to target molecules. For example, an excess of single reactive center reagents having oligonucleotide probes attached thereto is used to analyze and/or label target nucleic acids. In some embodiments, once the nucleic acids have hybridized to each other, free unbound reagents are removed. Following this, intensity of fluorescence or number of detected fluorescent particles in the sample is measured (in the case of a fluorescently labeled reagent). This in turn allows detection of the target and determination of its concentration.

In some embodiments, two reagents each having a unique and distinct detectable label from the other can be used. For example, two quantum dots of different colors can be conjugated two different oligonucleotides. If these probes recognize different target sites on the same nucleic acid, a coincidence analysis can be used for the detection and identification of the target (see references in (Heinze et al. 2002)). In this case, the removal of unbound reagents is not necessary. Furthermore, the ability to use multiple colors, allows multiplexing of different assays. For example, reagents with 4 colors allow detection of 6 different targets using coincidence analysis.

Another way of analyzing binding of the reagents to a target without an intermediate clean up step in which unbound reagents are removed prior to analysis involves the use of FRET. In this case, a donor and an acceptor fluorophore must be used. Quantum dots are generally suitable donors for the energy transfer.

Quantum dots or quantum nanocrystals are comprised of small semiconductor particles having diameters in the range of several nanometers. Quantum dots are fluorescent, stable in solution, have low inherent non-specific binding to biological molecules, and have been successfully used in many cell-related (Jaiswal et al. 2003; Wu et al. 2003) and whole organism applications (Larson et al. 2003). The quantum dots used in these applications have multiple reaction groups, which make them less amenable to use in single molecule applications such as those described herein.

Quantum dots absorb light of virtually any wavelength and then rapidly emit the light in a different color of higher wavelength (and correspondingly lower energy). Their optical properties that can be readily customized by changing their size or composition. Thus, it is possible to adjust absorption and emission wavelengths by changing the dot size (i.e., different sized quantum dots emit light of different wavelengths). For example, 3 nm CdSe quantum dots emit at 520 nm and 5.5 nm CdSe quantum dots emit at 630 nm. It is to be understood that quantum dots of intermediates sizes will emit in intermediate wavelengths. It is also to be understood that even within a population of quantum dots that are presumably homogeneous in size, there will be some size variability that is expected to mimic a Gaussian distribution. Quantum dots have been described in at least U.S. Pat. No. 6,207,392, the entire contents of which are incorporated by reference herein.

Optical properties of quantum dots can be modulated by electric field (Wang et al. 2001), and thus their fluorescent emission can be induced not only by light radiation (e.g., via lasers, lamps, LEDs, etc.) but also electronically (Colvin et al. 1994; Ding et al. 2002).

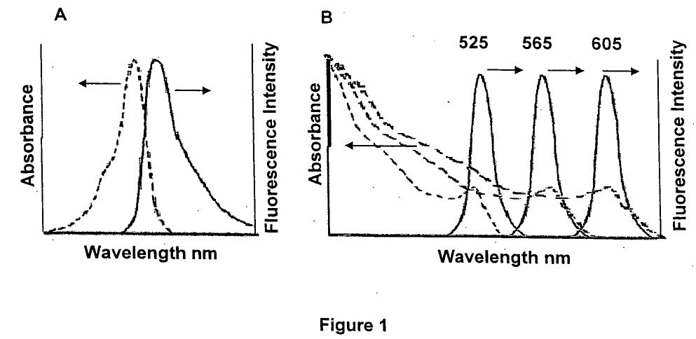

Quantum dots are capable of absorbing light of wavelengths less than their emission spectra. For example, quantum dots that emit at a maximum spectrum of 520 nm can absorb wavelengths up to 519 nm (as shown in FIG. 1B). Quantum dots that emit at longer wavelengths are able to absorb correspondingly longer wavelengths up to but not greater than their emission spectrum.

The general structure of a quantum dot consists of a core and a shell. The core is generally composed of cadmium selenide (CdSe), cadmium telluride (CdTe) or indium arsenide (InAs). CdSe provides emission in the visible range (i.e., about 500-750 nm), CdTe provides emission in the red to near infrared range (i.e., 560-700 nm), and InAs provides emission in the near infrared (NIR) range (i.e., about 700-2000 nm). InP/InAs quantum dots with an extra SiO₂ shell provide emission in the 400-2000 nm range. Emission wavelengths up through and including the 1800 nm range can also be achieved with quantum dots comprising different semiconductors, such as but not limited to PbSe.

The outer shell of quantum dot protects and insulates the core from environmental effects, amplifies optical properties, and provides a novel surface coating that enables derivatization of reactive groups. The reactive group as stated above (e.g., streptavidin reactive groups, biotin, antibody reactive groups, antigens, lectins, nucleic acids, and the like) can be any group that interacts with other molecules either reversibly or irreversibly and preferably with high affinity (e.g., affinity constants on the order of 10⁸, 10⁹, 10¹⁰, 10¹¹, 10¹², 10¹³, 10⁴, or 10¹⁵ M⁻¹).

Properties of quantum dots have been discussed elsewhere (Alivisatos 1996a; Alivisatos 1996b). The spectral properties of quantum dots (Alivisatos 1996b) differ significantly from those of organic fluorophores (Haugland 2002). FIGS. 1A and 1B illustrate spectra for organic fluorophores and quantum dots, respectively. Excitation (i.e., absorption) and emission spectra of an organic fluorophore are asymmetric and approximate mirrors of each other. A typical emission spectrum width (i.e., full width at half-height, FWHH) of an organic fluorophore is about 50-70 nm, while its excitation spectrum width is typically 10-30% narrower. Therefore, every organic fluorophore can be excited only within a narrow spectral range. The wavelength range of the spectrum and its shape are generally determined by the chemical structure of the organic fluorophore and its surroundings. Fluorophores with different chemical structures are used (and/or needed) to ensure emission in different spectral ranges. Only about 3-4 organic fluorophores can be detected without overlapping with an emission spectrum of another organic fluorophore, within the optimal sensitivity range of a typical photodetector.

The maximum emission wavelength of a quantum dot on the other hand is determined by the size of the quantum dot. For example, CdSe quantum dots having diameters of 2.1 and 4.6 nm emit at 480 and 660 nm, respectively (Alivisatos 1996b). Unlike organic fluorophores, all quantum dots can be excited within any given spectral range, although the excitation efficiency increases for shorter wavelengths (FIG. 1B). Emission spectra of quantum dots are generally symmetric and narrower than the emission spectra of organic fluorophores (e.g., FWHH is typically 25-35 nm). Therefore, many different quantum dots can be excited at the same excitation wavelength, 6-8 quantum dots can be detected without overlapping emission spectra (within the optimal sensitivity range of a photodetector), and it is possible to produce engineer quantum dots corresponding to every emission wavelength in the near UV to IR range. Additionally, quantum dots are brighter and more photostable than organic fluorophores (Alivisatos 1996a; Alivisatos 1996b).

As discussed herein, the invention embraces detectable labels such as fluorescent quantum dots as well as other labels. These other labels can take various forms and thereby perform various functions in the aspects and embodiments described herein. For example, multiple reactive center reagents that are not inherently detectable can be made so by conjugating to them detectable labels such as those described herein. As an example, if the reagent has streptavidin reactive groups, it can be made detectable by saturating virtually all streptavidin reactive groups with a detectably labeled biotin rather than an unconjugated biotin (as described above for inherently detectable quantum dots). As another example, detectable single reactive center reagents conjugated to a target specific probe can be used together with another target specific detectable probe to analyze a biological molecule such as a nucleic acid. The second probe may be labeled with any detectable label including organic fluorophores. In some instances, analysis of the biological molecule will require coincident detection of signals from both probes.

In other instances, the analysis will require coincident and sufficiently proximal presence of both probes on a biological molecule to allow FRET to occur. If FRET based analysis is performed with a quantum dot, then usually the second label will be something other than a quantum dot, such as for example an organic fluorophore. As will be understood by those of ordinary skill in the art, FRET requires a donor fluorophore and an acceptor fluorophore. The donor fluorophore absorbs the excitation light and then emits light of a longer wavelength that falls within the excitation range of the acceptor fluorophore. When the donor and acceptor fluorophores are located within a sufficient distance of each other (e.g., within 2-20 nucleotides distance of each other, or within about 6.8-68 Angstroms of each other). Preferably, the distance is one that enables at least 50% energy transfer efficiency, more preferably at least 65% energy transfer efficiency and most preferably at least 70% energy transfer efficiency. FRET generally requires only one excitation source (and thus wavelength) and sometimes only one detector. If a single detector is used, it is generally set to either the emission spectrum of the donor or acceptor fluorophore. It is set to the donor fluorophore emission spectrum if FRET is detected by quenching of donor fluorescence. Alternatively, it is set to the acceptor fluorophore emission spectrum if FRET is detected by acceptor fluorophore emission. In some embodiments, FRET emissions of both donor and acceptor fluorophores can be detected. In still other embodiments, the donor is excited with polarized light and polarization of both emission spectra is detected.

The nature of the detectable labels to be used in generating single reactive center reagents or for labeling other probes will depend upon the excitation source and detector available. In some embodiments, fluorophores whether quantum dots or organic fluorophores are preferred, particularly where FRET based analysis is envisioned.

A detectable label is a moiety, the presence of which can be ascertained directly or indirectly. Generally, detection of the label involves the creation of a detectable signal such as for example an emission of energy. The label can be detected directly for example by its ability to emit and/or absorb electromagnetic radiation of a particular wavelength. A label can be detected indirectly for example by its ability to bind, recruit and, in some cases, cleave another moiety which itself may emit or absorb light of a particular wavelength (e.g., an epitope tag such as the FLAG epitope, an enzyme tag such as horseradish peroxidase, etc.). Generally the detectable label can be selected from the group consisting of directly detectable labels such as a fluorescent molecule (e.g., fluorescein, rhodamine, tetramethylrhodamine, R-phycoerythrin, Cy-3, Cy-5, Cy-7, Texas Red, Phar-Red, allophycocyanin (APC), fluorescein amine, eosin, dansyl, umbelliferone, 5-carboxyfluorescein (FAM), 2′7′-dimethoxy-4′5′-dichloro-6-carboxyfluorescein (JOE), 6 carboxyrhodamine (R6G), N,N,N′,N′-tetramethyl-6-carboxyrhodamine (TAMRA), 6-carboxy-X-rhodamine (ROX), 4-(4′-dimethylaminophenylazo)benzoic acid (DABCYL), 5-(2′-aminoethyl) aminonaphthalene-1-sulfonic acid (EDANS), 4-acetamido-4′-isothiocyanatostilbene-2,2′disulfonic acid, acridine, acridine isothiocyanate, r-amino-N-(3-vinylsulfonyl)phenylnaphthalimide-3,5, disulfonate (Lucifer Yellow VS), N-(4-anilino-1-naphthyl)maleimide, anthranilamide, Brilliant Yellow, coumarin, 7-amino-4-methylcoumarin, 7-amino-4-trifluoromethylcouluarin (Coumarin 151), cyanosine, 4′, 6-diaminidino-2-phenylindole (DAPI), 5′, 5″-diaminidino-2-phenylindole (DAPI), 5′, 5″-dibromopyrogallol-sulfonephthalein (Bromopyrogallol Red), 7-diethylamino-3-(4′-isothiocyanatophenyl)-4-methylcoumarin diethylenetriamine pentaacetate, 4, 4′-diisothiocyanatodihydro-stilbene-2,2′-disulfonic acid, 4,4′-diisothiocyanatostilbene-2,2′-disulfonic acid, 4-dimethylaminophenylazophenyl-4′-isothiocyanate (DABITC), eosin isothiocyanate, erythrosin B, erythrosin isothiocyanate, ethidium, 5-(4,6-dichlorotriazin-2-yl)aminofluorescein (DTAF), QFITC (XRITC), fluorescamine, IR144, IR1446, Malachite Green isothiocyanate, 4-methylumbelliferone, ortho cresolphthalein, nitrotyrosine, pararosaniline, Phenol Red, B-phycoerythrin, o-phthaldialdehyde, pyrene, pyrene butyrate, succinimidyl 1-pyrene butyrate, Reactive Red 4 (Cibacron. RTM. Brilliant Red 3B-A), lissamine rhodamine B sulfonyl chloride, rhodamine B, rhodamine 123, rhodamine X, sulforhodamine B, sulforhodamine 101, sulfonyl chloride derivative of sulforhodamine 101, tetramethyl rhodamine, riboflavin, rosolic acid, and terbium chelate derivatives), a chemiluminescent molecule, a bioluminescent molecule, a chromogenic molecule, a radioisotope (e.g., P³² or H³, ¹⁴C, ¹²⁵I and ¹³¹I), an electron spin resonance molecule (such as for example nitroxyl radicals), an optical or electron density molecule, an electrical charge transducing or transferring molecule, an electromagnetic molecule such as a magnetic or paramagnetic bead or particle, a semiconductor nanocrystal or nanoparticle, a colloidal metal, a colloid gold nanocrystal, a nuclear magnetic resonance molecule, and the like.

The detectable label can also be selected from the group consisting of indirectly detectable labels such as an enzyme (e.g., alkaline phosphatase, horseradish peroxidase, β-galactosidase, glucoamylase, lysozyme, luciferases such as firefly luciferase and bacterial luciferase (U.S. Pat. No. 4,737,456); saccharide oxidases such as glucose oxidase, galactose oxidase, and glucose-6-phosphate dehydrogenase; heterocyclic oxidases such as uricase and xanthine oxidase coupled to an enzyme that uses hydrogen peroxide to oxidize a dye precursor such as HRP, lactoperoxidase, or microperoxidase), an enzyme substrate, an affinity molecule, a ligand, a receptor, a biotin molecule, an avidin molecule, a streptavidin molecule, an antigen (e.g., epitope tags such as the FLAG or HA epitope), a hapten (e.g., biotin, pyridoxal, digoxigenin fluorescein and dinitrophenol), an antibody, an antibody fragment, a microbead, and the like.

Fluorophore pairs are two fluorophores that are capable of undergoing FRET to produce or eliminate a detectable signal when positioned in proximity to one another. Examples of donors include Alexa488, Alexa546, BODIPY493, Oyster556, Fluor (FAM), Cy3 and TMR (Tamra). Examples of acceptors include Cy5, Alexa594, Alexa647 and Oyster656. Cy5 can work as a donor with Cy3, TMR or Alexa546, as an example. FRET should be possible with any fluorophore pair having fluorescence maxima spaced at 50-100 nm from each other.

The label may be of a chemical, lipid, carbohydrate, peptide or nucleic acid nature although it is not so limited. Those of ordinary skill in the art will know of other suitable labels for use in the invention.

Furthermore, conjugation of these labels to for example reactive groups and/or probes can be performed using standard techniques common to those of ordinary skill in the art. For example, U.S. Pat. Nos. 3,940,475 and 3,645,090 demonstrate conjugation of fluorophores and enzymes to antibodies.

As used herein, “conjugated” means two entities stably bound to one another by any physicochemical means. It is important that the nature of the attachment is such that it does not substantially impair the effectiveness of either entity. Keeping these parameters in mind, any covalent or non-covalent linkage known to those of ordinary skill in the art is contemplated unless explicitly stated otherwise herein. Noncovalent conjugation includes hydrophobic interactions, ionic interactions, high affinity interactions such as biotin-avidin and biotin-streptavidin complexation and other affinity interactions. Such means and methods of attachment are known to those of ordinary skill in the art.

The detection system will depend upon the type of detectable labels used. Therefore these roughly correlate with the detectable labels discussed herein. There is a number of detection systems known in the art and these include a fluorescent detection system, a confocal laser microscopy detection system, a near field detection system, a chemiluminescent detection system, a chromogenic detection system, a photographic or autoradiographic film detection system, an electrical detection system, a electromagnetic detection system, a charge coupled device (CCD) detection system, an electron microscopy detection system, an atomic force microscopy (AFM) detection system, a scanning tunneling microscopy (STM) detection system, a scanning electron microscopy detection system, an electron density detection system, a refractive index detection system such as a total internal reflection (TIR) detection system, an electron spin resonance (ESR) detection system, and a nuclear magnetic resonance (NMR) detection system.

The methods of the invention can be used to generate information about preferably biological molecules such as nucleic acids. The invention can however be used to analyze other naturally or non-naturally occurring molecules. This information is based on signals arising from the binding of probes to target molecules. In some instances, the information is unit specific information which refers any structural information about one, some, or all of the units that make up the biological molecule. If the biological molecule is a nucleic acid, the units are single or combinations of nucleotides, preferably arranged contiguously. The structural information obtained by analyzing a biological molecule may include the identification of its characteristic properties which (in turn) allows for, for example, the identification of its presence in or absence from a sample, determination of the relatedness of more than one biological molecules, identification of the size of the biological molecule, determination of the proximity or distance between two or more individual units within a biological molecule, determination of the order of two or more individual units within a biological molecule, and/or identification of the general composition of the biological molecule. Since the structure and function of biological molecules are interdependent, structural information can reveal important information about the function of the molecule.

The sensitivity of methods provided herein allows single polymers such as nucleic acids to be analyzed individually. Thus, the term “analyzing a biological molecule” as used herein means obtaining some information about the structure of the molecule such as its size, the order of its units, its relatedness to other molecules, the identity of its units, or its presence or absence in a sample. Analyzing the target generally requires contacting the single reactive center reagent(s) with a target and determining the binding pattern of the reagent(s) to the target. As stated herein, such binding pattern may be simply a determination of whether the reagent(s) is bound to the target. Alternatively, it may be a determination of the binding sites within the target (thereby providing a map of sites along the target). Levels of fluorescence as well as position of fluorescence may therefore be analyzed.

Analyzing a biological molecule applies to analyzing a biological polymer such as a nucleic acid or a peptide or protein. It is to be understood that the same definitions apply to non-naturally occurring molecules such as non-naturally occurring polymers.

The term “nucleic acid” refers to multiple linked nucleotides (i.e., molecules comprising a sugar (e.g., ribose or deoxyribose) linked to an exchangeable organic base, which is either a pyrimidine (e.g., cytosine (C), thymidine (T) or uracil (U)) or a purine (e.g., adenine (A) or guanine (G)). “Nucleic acid” and “nucleic acid molecule” are used interchangeably and refer to oligoribonucleotides as well as oligodeoxyribonucleotides. The terms shall also include polynucleosides (i.e., a polynucleotide minus a phosphate) and any other organic base containing nucleic acid. The nucleic acids may be single or double stranded. The nucleic acid being analyzed and/or labeled is referred to as the nucleic acid target.

Nucleic acid targets and nucleic acid probes may be DNA or RNA, although they are not so limited. DNA may be genomic DNA such as nuclear DNA or mitochondrial DNA. RNA may be mRNA, miRNA, rRNA and the like. Nucleic acids may be naturally occurring such as those recited above, or may be synthetic such as cDNA. In important embodiments, the nucleic acid is a genomic nucleic acid. In related embodiments, the nucleic acid is a fragment of a genomic nucleic acid. The size of the nucleic acid is not critical to the invention and it is generally only limited by the detection system used.

Harvest and isolation of nucleic acids are routinely performed in the art and suitable methods can be found in standard molecular biology textbooks. (See, for example, Maniatis' Handbook of Molecular Biology.) The nucleic acid may be harvested from a biological sample such as a tissue or a biological fluid. The term “tissue” as used herein refers to both localized and disseminated cell populations including but not limited, to brain, heart, breast, colon, bladder, uterus, prostate, stomach, testis, ovary, pancreas, pituitary gland, adrenal gland, thyroid gland, salivary gland, mammary gland, kidney, liver, intestine, spleen, thymus, bone marrow, trachea, and lung. Biological fluids include saliva, sperm, serum, plasma, blood and urine, but are not so limited. Both invasive and non-invasive techniques can be used to obtain such samples and are well documented in the art.

The methods of the invention may be performed in the absence of prior nucleic acid amplification in vitro. In some preferred embodiments, the nucleic acid is directly harvested and isolated from a biological sample (such as a tissue or a cell culture), without its amplification. Accordingly, some embodiments of the invention involve analysis of “non in vitro amplified nucleic acids”. As used herein, a “non in vitro amplified nucleic acid” refers to a nucleic acid that has not been amplified in vitro using techniques such as polymerase chain reaction or recombinant DNA methods.

A non in vitro amplified nucleic acid may, however, be a nucleic acid that is amplified in vivo (e.g., in the biological sample from which it was harvested) as a natural consequence of the development of the cells in the biological sample. This means that the non in vitro nucleic acid may be one which is amplified in vivo as part of gene amplification, which is commonly observed in some cell types as a result of mutation or cancer development.

In some embodiments, the invention embraces nucleic acid derivatives as targets and/or probes. As used herein, a “nucleic acid derivative” is a non-naturally occurring nucleic acid. Nucleic acid derivatives may contain non-naturally occurring elements such as non-naturally occurring nucleotides and non-naturally occurring backbone linkages. These include substituted purines and pyrimidines such as C-5 propyne modified bases, 5-methylcytosine, 2-aminopurine, 2-amino-6-chloropurine, 2,6-diaminopurine, hypoxanthine, 2-thiouracil and pseudoisocytosine. Other such modifications are well known to those of skill in the art.

The nucleic acids may also encompass substitutions or modifications, such as in the bases and/or sugars. For example, they include nucleic acids having backbone sugars which are covalently attached to low molecular weight organic groups other than a hydroxyl group at the 3′ position and other than a phosphate group at the 5′ position. Thus, modified nucleic acids may include a 2′-O-alkylated ribose group. In addition, modified nucleic acids may include sugars such as arabinose instead of ribose.

The nucleic acids may be heterogeneous in backbone composition thereby containing any possible combination of nucleic acid units linked together such as peptide nucleic acids (which have amino acid linkages with nucleic acid bases, and which are discussed in greater detail herein). In some embodiments, the nucleic acids are homogeneous in backbone composition.

As used herein with respect to linked units of a nucleic acid, “linked” or “linkage” means two entities bound to one another by any physicochemical means. Any linkage known to those of ordinary skill in the art, covalent or non-covalent, is embraced. Natural linkages, which are those ordinarily found in nature connecting the individual units of a particular nucleic acid, are most common. Natural linkages include, for instance, amide, ester and thioester linkages. The individual units of a nucleic acid analyzed by the methods of the invention may be linked, however, by synthetic or modified linkages. Nucleic acids where the units are linked by covalent bonds will be most common but those that include hydrogen bonded units are also embraced by the invention. It is to be understood that all possibilities regarding nucleic acids appear equally to nucleic acid targets and nucleic acid probes.

A nucleic acid target can be bound by one or more sequence specific probes. “Sequence specific” when used in the context of a probe for a nucleic acid target means that the probe recognizes a particular linear arrangement of nucleotides or derivatives thereof. In preferred embodiments, the probe is itself composed of nucleic acid elements such as DNA, RNA, PNA and LNA elements and combinations thereof (as discussed below). In preferred embodiments, the linear arrangement includes contiguous nucleotides or derivatives thereof that each bind to a corresponding complementary nucleotide in the probe. In some embodiments, however, the sequence may not be contiguous as there may be one, two, or more nucleotides that do not have corresponding complementary residues on the probe. The specificity of binding can be manipulated in a number of ways including temperature, salt concentration and the like. Those of ordinary skill in the art will be able to determine optimum conditions for a desired specificity.

It is to be understood that any molecule that is capable of recognizing a target nucleic acid with structural or sequence specificity can be used as a nucleic acid probe. In most instances, such probes will be themselves nucleic acid in nature. Also in most instances, such probes will form at least a Watson-Crick bond with the nucleic acid target. In other instances, the nucleic acid probe can form a Hoogsteen bond with the nucleic acid target, thereby forming a triplex. A nucleic acid probe that binds by Hoogsteen binding enters the major groove of a nucleic acid target and hybridizes with the bases located there. Examples of these latter probes include molecules that recognize and bind to the minor and major grooves of nucleic acids (e.g., some forms of antibiotics). In some embodiments, the nucleic acid probes can form both Watson-Crick and Hoogsteen bonds with the nucleic acid target. BisPNA probes, for instance, are capable of both Watson-Crick and Hoogsteen binding to a nucleic acid.

In some embodiments, the nucleic acid probe is a peptide nucleic acid (PNA), a bisPNA clamp, a pseudocomplementary PNA, a locked nucleic acid (LNA), DNA, RNA, or co-nucleic acids of the above such as DNA-LNA co-nucleic acids. In some instances, the nucleic acid target can also be comprised of any of these elements.

PNAs are DNA analogs having their phosphate backbone replaced with 2-aminoethyl glycine residues linked to nucleotide bases through glycine amino nitrogen and methylenecarbonyl linkers. PNAs can bind to both DNA and RNA targets by Watson-Crick base pairing, and in so doing form stronger hybrids than would be possible with DNA or RNA based probes.

PNAs are synthesized from monomers connected by a peptide bond (Nielsen, P. E. et al. Peptide Nucleic Acids, Protocols and Applications, Norfolk: Horizon Scientific Press, p. 1-19 (1999)). They can be built with standard solid phase peptide synthesis technology. PNA chemistry and synthesis allows for inclusion of amino acids and polypeptide sequences in the PNA design. For example, lysine residues can be used to introduce positive charges in the PNA backbone. All chemical approaches available for the modifications of amino acid side chains are directly applicable to PNAs.

PNA has a charge-neutral backbone, and this attribute leads to fast hybridization rates of PNA to DNA (Nielsen, P. E. et al. Peptide Nucleic Acids, Protocols and Applications, Norfolk: Horizon Scientific Press, p. 1-19 (1999)). The hybridization rate can be further increased by introducing positive charges in the PNA structure, such as in the PNA backbone or by addition of amino acids with positively charged side chains (e.g., lysines). PNA can form a stable hybrid with DNA molecule. The stability of such a hybrid is essentially independent of the ionic strength of its environment (Orum, H. et al., BioTechniques 19(3):472-480 (1995)), most probably due to the uncharged nature of PNAs. This provides PNAs with the versatility of being used in vivo or in vitro. However, the rate of hybridization of PNAs that include positive charges is dependent on ionic strength, and thus is lower in the presence of salt.

Several types of PNA designs exist, and these include single strand PNA (ssPNA), bisPNA and pseudocomplementary PNA (pcPNA).

The structure of PNA/DNA complex depends on the particular PNA and its sequence. Single stranded PNA (ssPNA) binds to single stranded DNA (ssDNA) preferably in antiparallel orientation (i.e., with the N-terminus of the ssPNA aligned with the 3′ terminus of the ssDNA) and with a Watson-Crick pairing. PNA also can bind to DNA with a Hoogsteen base pairing, and thereby forms triplexes with double stranded DNA (dsDNA) (Wittung, P. et al., Biochemistry 36:7973 (1997)).

Single strand PNA is the simplest of the PNA molecules. This PNA form interacts with nucleic acids to form a hybrid duplex via Watson-Crick base pairing. The duplex has different spatial structure and higher stability than dsDNA (Nielsen, P. E. et al. Peptide Nucleic Acids, Protocols and Applications, Norfolk: Horizon Scientific Press, p. 1-19 (1999)). However, when different concentration ratios are used and/or in presence of complimentary DNA strand, PNA/DNA/PNA or PNA/DNA/DNA triplexes can also be formed (Wittung, P. et al., Biochemistry 36:7973 (1997)). The formation of duplexes or triplexes additionally depends upon the sequence of the PNA. Thymine-rich homopyrimidine ssPNA forms PNA/DNA/PNA triplexes with dsDNA targets where one PNA strand is involved in Watson-Crick antiparallel pairing and the other is involved in parallel Hoogsteen pairing. Cytosine-rich homopyrimidine ssPNA preferably binds through Hoogsteen pairing to dsDNA forming a PNA/DNA/DNA triplex. If the ssPNA sequence is mixed, it invades the dsDNA target, displaces the DNA strand, and forms a Watson-Crick duplex. Polypurine ssPNA also forms triplex PNA/DNA/PNA with reversed Hoogsteen pairing.

BisPNA includes two strands connected with a flexible linker. One strand is designed to hybridize with DNA by a classic Watson-Crick pairing, and the second is designed to hybridize with a Hoogsteen pairing. The target sequence can be short (e.g., 8 bp), but the bisPNA/DNA complex is still stable as it forms a hybrid with twice as many (e.g., a 16 bp) base pairings overall. The bisPNA structure further increases specificity of their binding. As an example, binding to an 8 bp site with a probe having a single base mismatch results in a total of 14 bp rather than 16 bp.

Preferably, bisPNAs have homopyrimidine sequences, and even more preferably, cytosines are protonated to form a Hoogsteen pair to a guanosine. Therefore, bisPNA with thymines and cytosines is capable of hybridization to DNA only at pH below 6.5. The first restriction—homopyrimidine sequence only—is inherent to the mode of bisPNA binding. Pseudoisocytosine (J) can be used in the Hoogsteen strand instead of cytosine to allow its hybridization through a broad pH range (Kuhn, H., J. Mol. Biol. 286:1337-1345 1999)).

BisPNAs have multiple modes of binding to nucleic acids (Hansen, G. I. et al., J. Mol. Biol. 307(1):67-74 (2001)). One isomer includes two bisPNA molecules instead of one. It is formed at higher bisPNA concentration and has a tendency to rearrange into the complex with a single bisPNA molecule. Other isomers differ in positioning of the linker around the target DNA strands. All the identified isomers still bind to the same binding site/target.

Pseudocomplementary PNA (pcPNA) (Izvolsky, K. I. et al., Biochemistry 10908-10913 (2000)) involves two single stranded PNAs added to dsDNA. One pcPNA strand is complementary to the target sequence, while the other is complementary to the displaced DNA strand. As the PNA/DNA duplex is more stable, the displaced DNA generally does not restore the dsDNA structure. The PNA/PNA duplex is more stable than the DNA/PNA duplex and the PNA components are self-complementary because they are designed against complementary DNA sequences. Hence, the added PNAs would rather hybridize to each other. To prevent the self-hybridization of pcPNA units, modified bases are used for their synthesis including 2,6-diamiopurine (D) instead of adenine and 2-thiouracil (^{S}U) instead of thymine. While D and ^{S}U are still capable of hybridization with T and A respectively, their self-hybridization is sterically prohibited.

Locked nucleic acid (LNA) molecules form hybrids with DNA, which are at least as stable as PNA/DNA hybrids (Braasch, D. A. et al., Chem & Biol. 8(1):1-7(2001)). Therefore, LNA can be used just as PNA molecules would be. LNA binding efficiency can be increased in some embodiments by adding positive charges to it. LNAs have been reported to have increased binding affinity inherently.

Commercial nucleic acid synthesizers and standard phosphoramidite chemistry are used to make LNAs. Therefore, production of mixed LNA/DNA sequences is as simple as that of mixed PNA/peptide sequences. The stabilization effect of LNA monomers is not an additive effect. The monomer influences conformation of sugar rings of neighboring deoxynucleotides shifting them to more stable configurations (Nielsen, P. E. et al. Peptide Nucleic Acids. Protocols and Applications, Norfolk: Horizon Scientific Press, p. 1-19 (1999)). Also, lesser number of LNA residues in the sequence dramatically improves accuracy of the synthesis. Naturally, most of biochemical approaches for nucleic acid conjugations are applicable to LNA/DNA constructs.

The probes can also be stabilized in part by the use of other backbone modifications. The invention intends to embrace, in addition to the peptide and locked nucleic acids discussed herein, the use of the other backbone modifications such as but not limited to phosphorothioate linkages, phosphodiester modified nucleic acids, combinations of phosphodiester and phosphorothioate nucleic acid, methylphosphonate, alkylphosphonates, phosphate esters, alkylphosphonothioates, phosphoramidates, carbamates, carbonates, phosphate triesters, acetamidates, carboxymethyl esters, methylphosphorothioate, phosphorodithioate, p-ethoxy, and combinations thereof.

Other backbone modifications, particularly those relating to PNAs, include peptide and amino acid variations and modifications. Thus, the backbone constituents of PNAs may be peptide linkages, or alternatively, they may be non-peptide linkages. Examples include acetyl caps, amino spacers such as 0-linkers, amino acids such as lysine (particularly useful if positive charges are desired in the PNA), and the like. Various PNA modifications are known and probes incorporating such modifications are commercially available from sources such as Boston Probes, Inc.

The length of probe can also determine the specificity of binding. The energetic cost of a single mismatch between the probe and the nucleic acid target is relatively higher for shorter sequences than for longer ones. Therefore, hybridization of smaller nucleic acid probes is more specific than is hybridization of longer nucleic acid probes because the longer probes can embrace mismatches and still continue to bind to the target depending on the conditions. One potential limitation to the use of shorter probes however is their inherently lower stability at a given temperature and salt concentration. In order to avoid this latter limitation, bisPNA probes can be used to bind shorter target sequences with sufficient hybrid stability.

Another consideration in determining the appropriate probe length is whether the nucleic acid sequence to be detected is unique or not. If the method is intended only to sequence a target nucleic acid, then unique sequences may not be that important provided they are sufficiently spaced apart from each other to be able to detect signal from each * binding event separately from the others.

Notwithstanding these provisos, the nucleic acid probes of the invention can be any length ranging from at least 4 nucleotides long to in excess of 1000 nucleotides long. In preferred embodiments, the probes are 5-100 nucleotides in length, more preferably between 5-25 nucleotides in length, and even more preferably 5-12 nucleotides in length. The length of the probe can be any length of nucleotides between and including the ranges listed herein, as if each and every length was explicitly recited herein. Thus, the length may be at least 5 nucleotides, at least 10 nucleotides, at least 15 nucleotides, at least 20 nucleotides, or at least 25 nucleotides. It should be understood that not all residues of the probe need hybridize to complementary residues in the nucleic acid target. For example, the probe may be 50 residues in length, yet only 25 of those residues hybridize to the nucleic acid target. Preferably, the residues that hybridize are contiguous with each other. Similarly, the probe and any nucleic acids to which it binds including those conjugated to magnetic beads for clean-up purposes need not be of the same size.

The probes are preferably single stranded, but they are not so limited. For example, when the probe is a bisPNA it can adopt a secondary structure with the nucleic acid target resulting in a triple helix conformation, with one region of the bisPNA clamp forming Hoogsteen bonds with the backbone of the target and another region of the bisPNA clamp forming Watson-Crick bonds with the nucleotide bases of the target.

The nucleic acid probe hybridizes to a complementary sequence within the nucleic acid target. The specificity of binding can be manipulated based on the hybridization conditions. For example, salt concentration and temperature can be modulated in order to vary the range of sequences recognized by the nucleic acid probes.

The various reagents, reactive groups, and probes may in some instances include a linker molecule. These linkers can be any variety of molecules, preferably non-active, such as nucleotides or multiple nucleotides, straight or branched saturated or unsaturated carbon chains of carbon, phospholipids, and the like, whether naturally occurring or synthetic. Additional linkers include alkyl and alkenyl carbonates, carbamates, and carbamides.

A wide variety of linkers can be used, many of which are commercially available, for example, from sources such as Boston Probes, Inc. (now Applied Biosystems, Inc.). Linkers are not limited to organic linkers, and rather can be inorganic also (e.g., —O—Si—O—, or O—P—O—). Additionally, they can be heterogeneous in nature (e.g., composed of organic and inorganic elements). Essentially any molecule having the appropriate size restrictions and capable of being linked to the various components such as fluorophore and probe can be used as a linker. As used herein, the terms linker and spacer are used interchangeably.

Molecules such as but not limited to polymers may be analyzed using a single molecule analysis system (e.g., a single polymer analysis system). A single molecule detection system is capable of analyzing single molecules separately from other molecules. Such a system may be capable of analyzing single molecules either in a linear manner (i.e., starting at a point and then moving progressively in one direction or another) and/or, as may be more appropriate in the present invention, in their totality. In certain embodiments in which detection is based predominately on the presence or absence of a signal, linear analysis may not be required. However, there are other embodiments embraced by the invention which would benefit from the ability to linearly analyze molecules (preferably nucleic acids) in a sample. These include applications in which the sequence of the nucleic acid is desired.

A linear polymer analysis system is a system that analyzes polymers in a linear manner (i.e., starting at one location on the polymer and then proceeding linearly in either direction therefrom). As a polymer is analyzed, the detectable labels attached to it are detected in either a sequential or simultaneous manner. When detected simultaneously, the signals usually form an image of the polymer, from which distances between labels can be determined. When detected sequentially, the signals are viewed in histogram (signal intensity vs. time), that can then be translated into a map, with knowledge of the velocity of the polymer. It is to be understood that in some embodiments, the polymer is attached to a solid support, while in others it is free flowing. In either case, the velocity of the polymer as it moves past, for example, an interaction station or a detector, will aid in determining the position of the labels, relative to each other and relative to other detectable markers that may be present on the polymer.

Accordingly, the analysis systems useful in the invention may deduce the total amount of label on a polymer, and in some instances, the location of such labels. The ability to locate and position the labels allows these patterns to be superimposed on other genetic maps, in order to orient and/or identify the regions of the genome being analyzed.

An example of a suitable system is the GeneEngine™ (U.S. Genomics, Inc., Woburn, Mass.). The Gene Engine™ system is described in PCT patent applications WO98/35012 and WO00/09757, published on Aug. 13, 1998, and Feb. 24, 2000, respectively, and in issued U.S. Pat. 6,355,420 B1, issued Mar. 12, 2002. The contents of these applications and patent, as well as those of other applications and patents, and references cited herein are incorporated by reference in their entirety. This system is both a single molecule analysis system and a linear polymer analysis system. It allows, for example, single nucleic acids to be passed through an interaction station in a linear manner, whereby the nucleotides in the nucleic acid are interrogated individually in order to determine whether there is a detectable label conjugated to the nucleic acid. Interrogation involves exposing the nucleic acid to an energy source such as optical radiation of a set wavelength. The mechanism for signal emission and detection will depend on the type of label sought to be detected, as described herein.

Other single molecule nucleic acid analytical methods which involve elongation of DNA molecules can also be used in the methods of the invention. These include fiber-fluorescence in situ hybridization (fiber-FISH) (Bensimon, A. et al., Science 265(5181):2096-2098 (1997)). In fiber-FISH, nucleic acid molecules are elongated and fixed on a surface by molecular combing. Hybridization with fluorescently labeled probe sequences allows determination of sequence landmarks on the nucleic acid molecules. The method requires fixation of elongated molecules so that molecular lengths and/or distances between markers can be measured. Pulse field gel electrophoresis can also be used to analyze the labeled nucleic acid molecules. Pulse field gel electrophoresis is described by Schwartz, D. C. et al., Cell 37(1):67-75 (1984). Other nucleic acid analysis systems are described by Otobe, K. et al., Nucleic Acids Res. 29(22):E109 (2001), Bensimon, A. et al. in U.S. Pat. No. 6,248,537, issued Jun. 19, 2001, Herrick, J. et al., Chromosome Res. 7(6):409:423 (1999), Schwartz in U.S. Pat. No. 6,150,089 issued Nov. 21, 2000 and U.S. Pat. No. 6,294,136, issued Sep. 25, 2001. Other linear polymer analysis systems can also be used, and the invention is not intended to be limited to solely those listed herein.

Optical detectable signals are generated, detected and stored in a database. The signals can be analyzed to determine structural information about the nucleic acid. The signals can be analyzed by assessing the intensity of the signal to determine structural information about the nucleic acid. The computer may be the same computer used to collect data about the nucleic acids, or may be a separate computer dedicated to data analysis. A suitable computer system to implement embodiments of the present invention typically includes an output device which displays information to a user, a main unit connected to the output device and an input device which receives input from a user. The main unit generally includes a processor connected to a memory system via an interconnection mechanism. The input device and output device also are connected to the processor and memory system via the interconnection mechanism. Computer programs for data analysis of the detected signals are readily available from CCD (charge coupled device) manufacturers.

### EXAMPLES

### Example 1

### Preparation of Single Center Quantum Dots using Quenching of Oligonucleotide-Quantum Dot Binding at Early Stage

A 585 nm quantum dot conjugated to streptavidin (Quantum Dot Corp., Hayward, Calif.) and a biotinylated conjugate of a 20-mer oligonucleotide (Integrated DNA Technologies, Coralville, Iowa) complimentary to a sequence on the E. coli Spike 8 system was used. The same approach can be applied to any other streptavidin-coated quantum dot. The oligonucleotide has the following sequence: 5′-ACC AGT TTC TTC ACT GCC GC-sp18-BioTEG-3′ (SEQ ID NO: 1). TEG (tetra-ethyleneglycol) is a 15 atom long linker and Sp 18 is an 18 atom carbon spacer. The tether between the sequence and the biotin was prepared in this manner to reduce any potential steric inhibition of the hybridization to the target (RNA) by the bulky quantum dot.

Different amounts of the oligonucleotide (excess between 100× and 0.25×) were incubated with 5 nM solution of quantum dots for 1 hour. The samples were then analyzed with electrophoresis on a 2% agarose gel (loaded 10 ul of sample on gel-5 nM quantum dots). The quantum dots bound to oligonucleotide migrated faster in the gel than the free quantum dots. From this analysis the binding conditions were selected to produce a sample with high proportion of 1:1 oligonucleotide-quantum dot complex: the incubation with 4.5× excess of oligonucleotide was performed for 1 hour at room temperature. After this time, an excess of free biotin (it was 1000× excess to the quantum dots, which resulted in about ˜10×-2× excess over biotin-binding sites) was added.

The free quantum dots were removed using magnetic beads (NEB, Beverly, Mass.). The specified amount of streptavidin-coated beads was hybridized to a biotinylated oligonucleotide complimentary to the oligonucleotide on the quantum dot. This bead-bound complimentary oligonucleotide had the following sequence: 5′-Biotin/GTT TGA ACA AGGTG-3′(SEQ ID NO: 2). The short length of this oligonucleotide (14 base pairs) was selected because of its low melting temperature (45° C.). After addition of the specified amount of oligonucleotide (NEB catalog) to the beads, the mixture was mixed at room temperature for 1 hour. The beads were then pulled and pelleted with a magnet, and washed three times with 1×TE buffer solution, and finally resuspended in 1×TE buffer.

The beads were combined with the oligonucleotide/quantum dot mixture and incubated overnight at room temperature for hybridization. The beads were then pelleted, the supernatant removed and the beads washed several times. The mixture was then heated to 50° C. for 1 hour to denature the complex of the quantum dot-bound and bead-bound oligonucleotides and the components were separated from each other. The supernatant, containing the quantum dots bound to one oligonucleotide, was removed and saved.

References

Alivisatos, A. P. 1996a. Perspectives on the physical chemistry of semiconductor nanocrystals. J. Phys. Chem. 100: 13226-13239.
Alivisatos, A. P. 1996b. Semiconductor clusters, nanocrystals, and quantum dots. Science 271: 933-937.
Colvin, V. L., Schlamp, M. C., and Alivisatos, A. P. 1994. Nature 370: 354.
Ding, Z., Quinn, B. M., Haram, S. K., Pell, L. E., Korgel, B. A., and Bard, A. J. 2002. Electrochemistry and electrogenerated chemiluminescence from silicon nanocrystal quantum dots. Science 296: 1293-1297.
Haugland, R. P. 2002. Handbook of Fluorescent Probes and Research Products. Molecular Probes, Inc., Eugene, Oreg.
Heinze, K. G., Rarbach, M., Jahnz, M., and Schwille, P. 2002. Two-Photon Fluorescence Coincidence Analysis: Rapid Measurements of Enzyme Kinetics. Biophys. J. 83: 1671-1681.
Jaiswal, J. K., Mattouss, H., Mauro, J. M., and Simon, S. M. 2003. Long-term multiple color imaging of live cells using quantum dot bioconjugates. Nature Biotechnol. 21: 47-51.
Larson, D. R., Zipfel, W. R., Williams, R. M., Clark, S. W., Bruchez, M. P., Wise, F. W., and Webb, W. W. 2003. Water-soluble quantum dots for multiphoton fluorescence imaging in vivo. Science 300: 1434-1436.
Wang, C., Shim, M., and Guyot-Sionnest, P. 2001. Electrochromic nanocrystal quantum dots. Science 291: 2390-2392.
Wu, X., Liu, H., Liu, J., Haley, K. N., Treadway, J. A., Larson, J. P., Nianfeng, G., Peale, F., and Brochez, M. P. 2003. Immunofluorescent labeling of cancer marker Her2 and other cellular targets with semiconductor quantum dots. Nature Biotechnol. 21: 41-46.

### Equivalents

It should be understood that the preceding is merely a detailed description of certain embodiments. It therefore should be apparent to those of ordinary skill in the art that various modifications and equivalents can be made without departing from the spirit and scope of the invention, and with no more than routine experimentation. All references, patents and patent applications that are recited in this application are incorporated by reference herein in their entirety.

1. A method for producing a single reactive center reagent comprising contacting a multi reactive center reagent having a plurality of first reactive groups with
a) a probe conjugated to a second reactive group that is reactive to the first reactive group, and
b) unconjugated second reactive group, under conditions that favor binding of none or one conjugated probe per reagent.

2. The method of claim 1, wherein the multi reactive center reagent is inherently detectable.

3. The method of claim 2, wherein the multi reactive center reagent is a quantum dot or a fluorescent bead.

4. The method of claim 1, wherein the multi reactive center reagent is not inherently detectable.

5. The method of claim 4, wherein the multi reactive center reagent is a protein, a bead, or a particle.

6. The method of claim 1, wherein the multi reactive center reagent inherently comprises the plurality of first reactive groups.

7. The method of claim 1, wherein the multi reactive center reagent is derivatized to comprise the plurality of first reactive groups.

8. The method of claim 1, wherein the first reactive groups and second reactive groups are selected from the group consisting of biotin, streptavidin reactive groups, aptamers, aptamer ligands, receptors, receptor ligands, nucleic acids, enzymes, substrates, amines, carboxylic acids and esters.

9. The method of claim 1, wherein the first reactive group is biotin and the second reactive group is a streptavidin reactive group or an avidin reactive group.

10. The method of claim 1, wherein the first reactive group is a streptavidin reactive group or an avidin reactive group and the second reactive group is biotin.

11. The method of claim 1, wherein the first reactive group is an antigen or hapten and the second reactive group is an antibody reactive group.

12. The method of claim 1, wherein the first reactive group is an antibody reactive group and the second reactive group is an antigen or hapten.

13. The method of claim 1, wherein the first reactive group is a receptor and the second reactive group is a receptor ligand.

14-23. (canceled)

24. The method of claim 1, wherein the conditions that favor binding of none or one conjugated probe per reagent comprise excess unconjugated second reactive group.

25. (canceled)

26. The method of claim 1, wherein the conditions that favor binding of none or one conjugated probe per reagent comprise reducing binding time, increased temperature, or altered ion concentration.

27-47. (canceled)

48. A composition comprising
a single reactive center reagent as produced according to the method of claim 1.

49. A method for producing a single reactive center quantum dot comprising
contacting a streptavidin-conjugated quantum dot with a biotin-conjugated nucleic acid probe and unconjugated biotin, under conditions that favor binding of none or one biotin-conjugated nucleic acid probe per quantum dot.

50-52. (canceled)

53. A method for producing a single reactive center quantum dot comprising
contacting a biotin-conjugated quantum dot with a streptavidin-conjugated nucleic acid probe and unconjugated streptavidin, under conditions that favor binding of none or one streptavidin-conjugated nucleic acid probe per quantum dot.

54-78. (canceled)

79. A composition comprising
a single reactive center quantum dot as produced according to the method of claim 49.

80. A method for analyzing a target molecule comprising
contacting a target with the single reactive center reagent of claim 48, and
determining a binding pattern of the single reactive center reagent or the single reactive center quantum dot to the target.

81-90. (canceled)


SEQUENCE LISTING (2 sequences)

SEQ ID NO: 1 - DNA, 20 bp, Artificial Sequence
  oligonucleotide
accagtttcttcactgccgc

SEQ ID NO: 2 - DNA, 14 bp, Artificial sequence
  synthetic oligonucleotide
gtttgaacaaggtg